(19) United States
(12) Patent Application Publication
(10) Pub. No.: US 2019/0354200 A1
(43) Pub. Date: Nov. 21, 2019

(54) VIRTUAL FOOT PEDAL

(71) Applicant: Alcon Inc., Fribourg (CH)

(72) Inventor: Tobias Jura Rapoport, Berlin (DE)

(21) Appl. No.: 16/406,302

(22) Filed: May 8, 2019

Related U.S. Application Data
(60) Provisional application No. 62/672,307, filed on May 16, 2018.

(51) Int. Cl. G06F 3/033; G16H 40/63; G06F 3/01

(52) U.S. Cl. CPC G06F 3/0334; G06F 3/011; G06F 3/017; G16H 40/63

(57) ABSTRACT

A system for controlling a medical device with a virtual foot pedal is disclosed herein. The system may include at least one medical device, a display screen coupled to the medical device, and a virtual foot pedal. The virtual foot pedal may be connected to the medical device and may be further operable to capture motion in a tracking area. A method for controlling a medical console is disclosed herein. The method may include displaying one or more icons disposed on a display screen disposed on the medical console. The method may further include initiating the virtual foot pedal to emit a tracking area, monitoring movement of a foot in the tracking area, and receiving a selectin of at least one icon based on movement of the foot.

### BACKGROUND

Surgical operations often require many different and specialized tools. The tools may include a complex machine, designed to function and/or operate in a specific manner. Furthermore, tools may have individual controls that do not communicate and/or work with other controls. Specifically, controls may be in the form of foot pedals, which may allow a surgeon to operate and control a tool with their feet. This presents many challenges in the operating room during a surgical operation.

For example, there may be three or more foot pedals disposed at the foot of a surgeon. This may lead to tripping hazards and clog valuable space a surgeon may need during a complex surgical operation. Additionally, large numbers of foot pedals may become confusing to a surgeon during the operation. For example, a surgeon may accidentally use a controller for the wrong surgical tool, which may be problematic in the middle of a surgical operation. Current techniques for controlling surgical tools during a surgical operation are both rudimentary and onerous and may lead to costly mistakes that a patient may bear.

### SUMMARY

In an exemplary aspect, the present disclosure is directed to a system. The system may include at least one medical device, a display screen coupled to the medical device, and a virtual foot pedal. The virtual foot pedal may be connected to the medical device and may be further operable to capture motion in a tracking area.

In another exemplary aspect, the present disclosure is directed to a method for controlling a medical console. The method may include displaying one or more icons disposed on a display screen disposed on the medical console. The method may further include initiating the virtual foot pedal to emit a tracking area, monitoring movement of a foot in the tracking area, and receiving a selectin of at least one icon based on movement of the foot.

The different aspects may include on or more of the following features. The tracking area may comprise at least one zone and the at least one zone controls the at least one medical device. The display screen may be a heads up display and comprises at least one icon that controls the at least one medical device and the at least one icon may be operable to access sub-modes, wherein the sub-modes are operable to control functionality of the at least one medical device. The medical device may further comprise a console, wherein the virtual foot pedal may be wirelessly connected to the console. The medical device may further comprise a console, wherein the virtual foot pedal may be wired to the console. The virtual foot pedal may comprise a camera, wherein the camera may comprise a light source, wherein the light source may be pulsated. The virtual foot pedal may comprise a camera and markers, wherein the markers disposed on a foot, wherein the camera may be operable to track the movement of the markers in the tracking area. The virtual foot pedal comprises an infrared light source, wherein the infrared light source may be operable to emit an infrared light into the tracking area. Furthermore, the camera may be operable to sense visible light. The virtual foot pedal may further comprise a body and the body may be capable of stabilizing the virtual foot pedal and the body may further comprise a transmitter and a receiver. The transmitter may be operable to emit a sound wave into the tracking area. The receiver may be operable to sense a reflected wave from the tracking area. Additionally, the transmitter may be operable to emit an electromagnetic field into the tracking area. The virtual foot pedal may further comprises magnets and the magnets may be positionable in the tracking area and the receiver may be operable to sense alterations to the electromagnetic field due to the magnets.

Furthermore the method may comprise operating a medical device with the foot and wherein the virtual foot pedal may attached to the medical console wirelessly. At least one icon may be operable to access sub-modes and the sub-modes may be operable to control functionality of the medical console.

In another exemplary aspect, the present disclosure is directed to It is to be understood that both the foregoing general description and the following drawings and detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following.

### BRIEF DESCRIPTION OF THE DRAWINGS

These drawings illustrate examples of certain aspects of some of the embodiments of the present disclosure and should not be used to limit or define the disclosure.

FIG. 1 illustrates an example of a medical console and position tracking device.

FIG. 2 illustrates an example of an optical tracking device.

FIG. 3 illustrates another example of an optical tracking device.

FIG. 4 illustrates an example of an acoustic tracking device.

FIG. 5 illustrates an example of a magnetic tracking device.

FIG. 6 illustrates an example of an infrared tracking device.

FIG. 7 illustrates a schematic layout of a medical console and position tracking device.

FIG. 8 illustrates an example of a display screen.

FIG. 9 illustrates a medical device control method.

### DETAILED DESCRIPTION

For the purposes of promoting an understanding of the principles of the present disclosure, reference will now be made to the implementations illustrated in the drawings and specific language will be used to describe them. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with reference to one or more implementations may be combined with the features, components, and/or steps described with reference to other implementations of the present disclosure. For simplicity, in some instances the same reference numbers are used throughout the drawings to refer to the same or like parts.

The present disclosure generally relates to surgical instrument controls for use in an operating room for surgical operations. It should be noted that the system disclosed below may be utilized in any type of operation room for any type of medical treatment, including ophthalmic surgical procedures. Due to the nature of the complex workflow during an operation, hands-free control may be desirable. In embodiments, operations may include different hands-free input devices, such as virtual foot pedals. A virtual foot pedal may be defined as a device free of mechanical hardware that may be able to remotely detect the position of an operator. In examples, the virtual foot pedal may detect the movement of an operator's appendage, for example a foot, a hand, a leg, and/or an arm. Without limitation, the virtual foot pedal may be able to detect and track multiple movements of multiple appendages as well as the body of an operator. Each virtual foot pedal may control any number of medical devices in the operating room. Mechanical foot pedals may be limited by the number of buttons, joysticks, and/or rockers which may control identified functions on a specific device. Currently, control options may be limited by the number of available buttons and actions that may be assigned to each button disposed on a mechanical foot pedal. For example, a button may perform only a single action or at most a few context sensitive options. Thus, a complex sequence may be difficult to implement.

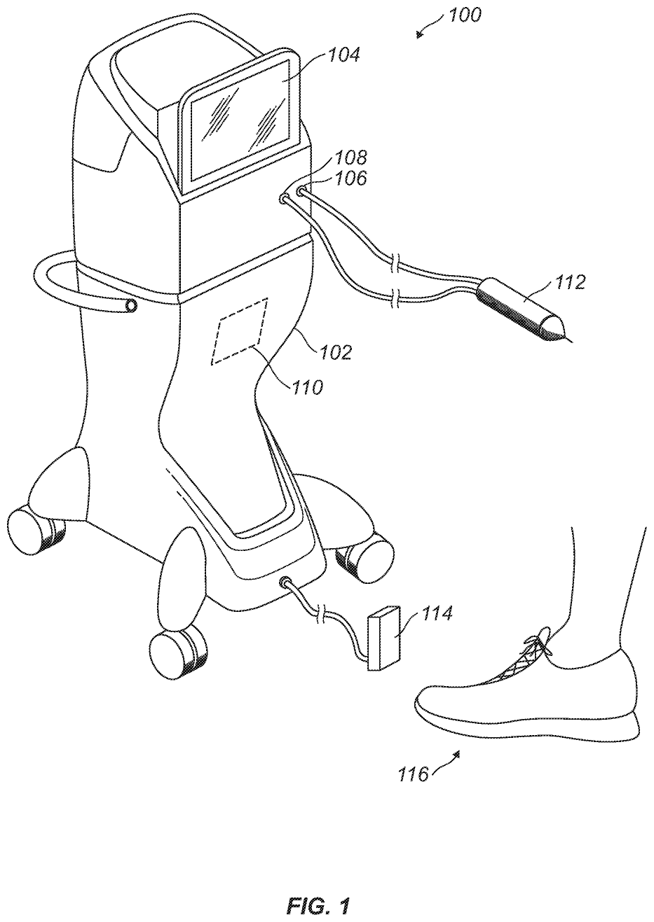

FIG. 1 illustrates a medical device 100 in accordance with example embodiments. However, without limitation, any type of surgical system may be used in embodiments disclosed below. In embodiments, medical device 100 may include a medical console 102 and an instrument 112. Instrument 112 may be any of a variety of medical instruments that may be used in medical applications, such as ophthalmic surgical procedures, including but not limited to, an ophthalmic microscope, an ultrasonic handpiece, a surgery guidance system, an intraoperative diagnostic unit, a vitrectomy instrument, an infusion cannula, intraocular lens (IOL) inserters, a trocar cannula, laser instrumentation, illumination proper (e.g., a chandelier lighting system, an endoilluminator, etc.). In the illustrated embodiment, the instrument 112 may be in the form of an IOL insert that is hydraulically driven. As illustrated, medical console 102 may include a display screen 104, an irrigation port 106, an aspiration port 108, and a position tracking device 114. Position tracking device 114 may be used in place of a foot pedal and may allow an operator to control the function of attached device without physical switches, buttons, triggers, touchscreen elements, keyboards, mice, and others. In embodiments, medical console 102 may be designed to be mobile and may be used by a user, such as a health care provider, to perform ophthalmic surgical procedures. Medical console 102 may also include a control system 110 that may be configured to process, receive, and store data to perform various functions associated with medical device 100.

Display screen 104 may communicate information to the user, and in some implementations, may show data relating to system operation and performance during a surgical procedure. In some embodiments, display screen 104 may be a touchscreen that allows the operator to interact with medical console 102 through a graphical user interface. Additionally, in other embodiments, display screen 104 may be a heads up display. A heads up display may be a transparent display that may present data without requiring an operator to look away from the operator's viewing area.

In some embodiments, medical console 102 may include various fluid handling systems for use during various ophthalmic surgical procedures. In the illustrated embodiment, medical console 102 may provide irrigation fluid through irrigation port 106. Medical console 102 may include a pump that can create a vacuum or suction force that may aspirate fluid and tissue through aspiration port 108. In some embodiments, the instrument 112 may use these or other fluid handling systems to drive the instrument 112. Specifically, the instrument 112 may be connected to irrigation port 106 through an irrigation line and may be connected to aspiration port 108 through an aspiration line. While the preceding description is directed to medical console 102 being configured for use with instrument 112 in the form of an IOL inserter, it should be understood that the present disclosure should encompass other configurations of medical console 102 depending, for example, on the particular application.

Position tracking device 114 may be described as a “virtual foot pedal.” A virtual foot pedal may be defined as any mechanism that may capture movement of a foot for operation of a device without mechanical movements. For example, currently a foot pedal may perform mechanical movement through physical switches, buttons, triggers, touchscreen elements, keyboards, mice, and others. Each one of these mechanical movements may control a function and/or operation of a device attached to the foot pedal. Foot pedals may be limited by the number of mechanical movements that may be disposed on the foot pedal. Thus, during operations any number of foot pedals may be utilized to control any number of specific and individual devices. Large numbers of foot pedals in an operation may become cumbersome and may lead accidental use of foot pedals that may be in close proximity to each other. Due to space limitations, large numbers of foot pedals may not be feasible. Additionally, foot pedals may be limited by the number of functions they may be able to perform. Thus, to perform more functions may require larger number of foot pedals. A single device, such as position tracking device 114, may declutter the operating room and may prevent unintended function and operation of devices.

In embodiments, position tracking device 114 may track the movement of at least a portion of an operator's foot 116. It should be noted that position tracking device 114 may track the movement of at least a portion of any part of the body designed to work with position tracking device 114. Without limitation, position tracking device 114 may register the position and/or movement of operator's foot 116 through the recognition of the rotation (pitch, yaw, and roll) of foot 116. Measuring the positions and/or movement of foot 116 may allow medical device to identify and determine operation and function of a device. For example, the position of foot 116 and a pre-determined movement of foot 116 in that position may operate and function a first device. A second position of foot 116 and a second pre-determined movement of foot 116 may operate and function a second device.

This may allow position tracking device 114 to control any number of devices and perform any number of functions and/or operations on those devices. Utilizing position tracking device 114 to control any number of device and perform any number of function and/or operations may declutter the operating room and may prevent the accidental use of devices during a surgical operation. Position tracking device 114 may be able to control the function and/or operation of other devices through position tracking. Position tracking may be performed by any number of sensors. Without limitation, position tracking sensors may include optical sensors, acoustic sensors, magnetic sensors, and/or thermal sensors. It should be noted that different types of sensors may work together in a system to form position tracking device 114. Position tracking sensors may also include many different individual devices that may work together to track the movement of foot 116.

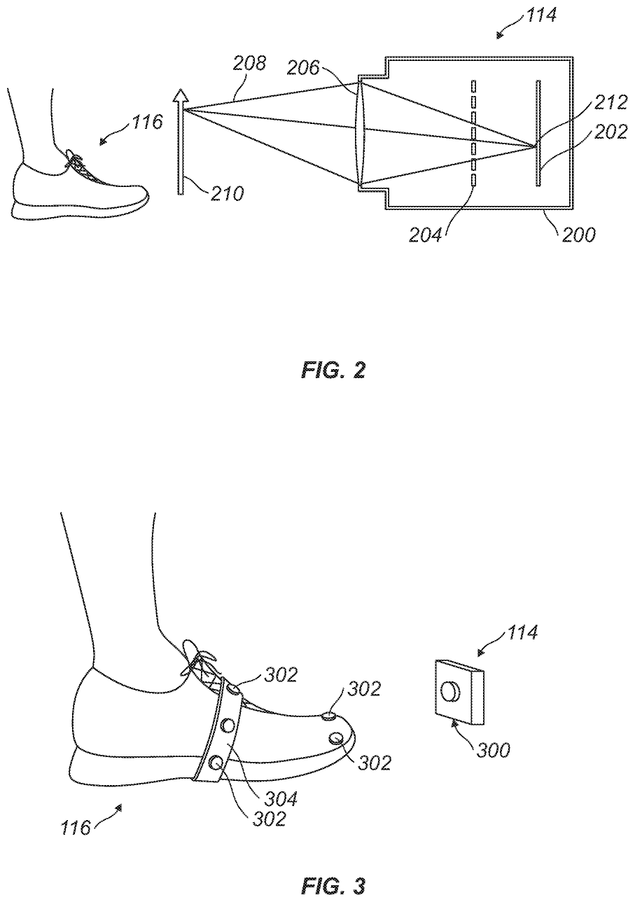

FIGS. 2 and 3 illustrate two different devices for optical tracking in accordance with embodiments of the present disclosure. In FIG. 2, position tracking device 114 may include a camera 200 to track the position and movement of foot 116. Disposed in camera 200 may be a sensor 202. Sensor 202 may be sensitive to the movement of light across its face. Additionally, camera 200 may comprise a mask 204. Mask 204 may be disposed within camera 200, as illustrated in FIG. 2, disposed along sensor 202, and/or along lens 206. During operations, camera 200 may be exposed to light 208 reflected off foot 116. As foot 116 moves along any axis 210, light 208 reflected from foot 116 may pass through lens 206 and into camera 200. It should be noted that light 208 may also pass through mask 204, which may be disposed at any suitable location in camera 200, as disclosed above. Light 208 passing through lens 206 is deflected and is disposed at a focal point 212 on sensors 202. During movement of foot 116, light 116 may be reflected into camera 200 at different angles. As the angle changes, focal point 212 may move along the face of sensors 202. As the focal point 212 moves, the movement of foot 116 may be tracked and/or recorded on medical device 100 (e.g., referring to FIG. 1). This type of optical tracking may be defined as marker-less tracking and/or passive tracking.

FIG. 3 illustrates optical tracking that may be defined as marker tracking and/or active tracking in accordance with embodiments of the present disclosure. In embodiments, position tracking device 114 may include camera 300 that may be designed to record the position and/or movement of markers 302. Markers 302 may comprise any material suitable to reflect visible light and/or infrared light, which may be captured and/or recorded by camera 300. In embodiments, makers 302 may emit visible light and/or infrared light, which may also be captured and/or recorded by camera 300. In examples, there may be at least one marker 302 disposed on foot 116. Without limitation, markers 302 may be disposed on foot 116 at any suitable location and/or may attach to foot 116 through any suitable connector 304. During operation, camera 300 may be disposed in any suitable location to view, capture, and/or record markers 302. Before a surgical operation may begin, camera 300 may calibrate in reference to foot 116 to determine a starting position. During surgical operations, as foot 116 moves, camera 300 may capture, record, and/or track the movement of markers 302 from a first position to a second position. The captured movements of markers 302, which in turn is foot 116, may be tracked and/or recorded on medical console 102 (e.g., referring to FIG. 1).

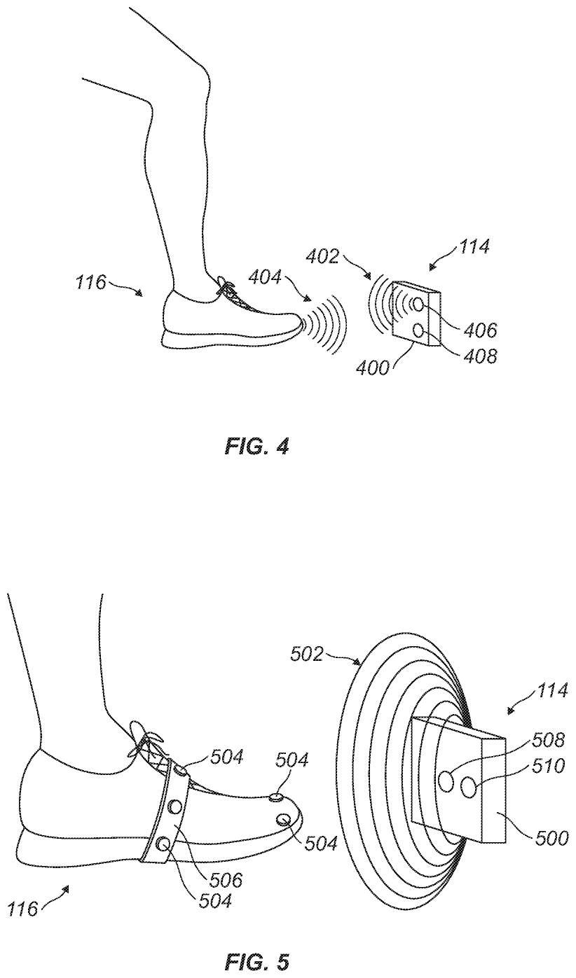

FIG. 4 illustrates an acoustic tracking device 400 in accordance with embodiments of the present disclosure. In embodiments, position tracking device 114 may include acoustic tracking device 400. There may be any number of suitable acoustic tracking devices 400 operating and/or functioning together to determine the movement and/or position of foot 116. Acoustic tracking device 400 may operate by emitting low frequency and/or high frequency sound waves 402, which may not be heard by humans. In embodiments, different types of sound waves 402 may be emitted concurrently and/or in a pre-determined structure by acoustic tracking device 400. Sound waves 402 may be emitted by a speaker 406 disposed at any suitable location on acoustic tracking device 400. As the sound waves 402 strike foot 116, reflected waves 404 may be sent back toward acoustic tracking device 400 and recorded. As illustrated, acoustic tracking device 400 may include one or more sensors 408 for recording reflected waves 404. Reflected wave s404 may be considered an echo. As foot 116 moves, reflected waves 404 may be altered due to the distance and location of foot 116 in relation to acoustic tracking device 400. This may allow acoustic tracking device 400 to determine the location and position of foot 116. The movement of foot 116 may be tracked and/or recorded on medical console 102 (e.g., referring to FIG. 1).

FIG. 5 illustrates a magnetic tracking device 500 in accordance with embodiments of the present disclosure. In embodiments, position tracking device 114 may include a magnetic tracking device 500. There may be any number of suitable magnetic tracking devices 500 operating and/or functioning together to determine the movement and/or position of foot 116. Magnetic tracking device 500 may operate by emitting an electromagnetic field 502 from a transmitter 508, which may be disposed on or in magnetic tracking device 500. Before operations, magnetic tracking device 500 may be calibrated to determine a base for electromagnetic field 502. A receiver 510 disposed on or in magnetic tracking device 500, may be able to sense electromagnetic field 502 and changes in electromagnetic field 502. In embodiments, foot 116 may comprise magnets 504. Magnets 504 may alter electromagnetic field 502. Without limitation, magnets 504 may be disposed individually on foot 116 and/or on a separate holding apparatus 506, which may attach to foot 116 in any suitable manner. In the illustrated embodiment, holding apparatus 506 is in the form of a band. By altering electromagnetic field 502 with magnets 504, the receiver 510 may be able to determine the location and/or position of foot 116. As foot 116 moves, electromagnetic field 502 may be altered due to the distance and location of foot 116 in relation to magnetic tracking device 500. It should be noted that magnets may not be used to alter electromagnetic field 502. Foot 116 may alter electromagnetic field 502 individually, for example, due to iron and water disposed within blood, or any other type of metal item, device, material, or the like disposed on foot 116 may alter electromagnetic field 502 without the use of magnets 504. This may allow magnetic tracking device 500 to determine the location and position of foot 116. The movement of foot 116 may be tracked and/or recorded on medical console 102 (e.g., referring to FIG. 1).

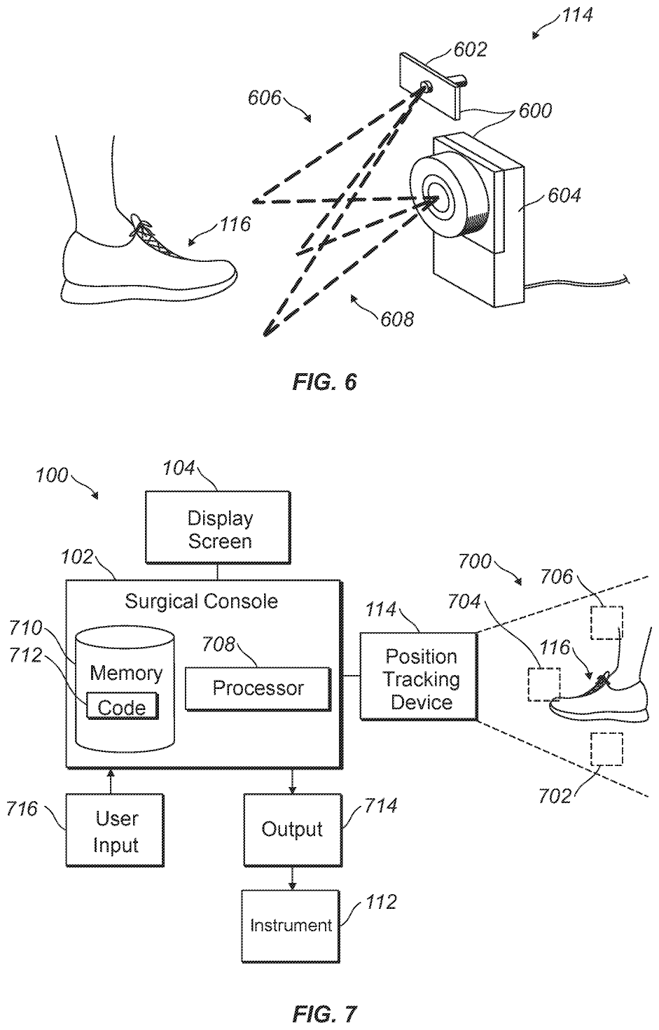

FIG. 6 illustrates an infrared tracking device 600 in accordance with embodiments of the present disclosure. In embodiments, position tracking device 114 may include an infrared tracking device 600. In embodiments, infrared tracking device 600 may comprise a light source 602 and a camera 604. Infrared tracking device 600 may function by emitting non-visible light 606 from light source 602. Non-visible light 606 may comprise infrared wavelengths on the light spectrum. This may allow infrared tracking device 600 to operate in a dark room and/or a lighted room. During operations, light source 602 may emit non-visible light 606 into a designated area. Foot 116 may be disposed in the path of non-visible light 606, which may produce a reflected light 608. Reflected light 608 may be recorded by camera 604. As foot 116 moves from one position to a second position, reflected light 608 may be altered and recorded by camera 604. This may allow infrared tracking device 600 to determine the locations and position of foot 116. The movement of foot 116 may be tracked and/or recorded on medical device 100 (e.g., referring to FIG. 1).

FIG. 7 illustrates an example of a medical device 100 for recording and/or tracking the movement and/or position of foot 116 with position tracking device 114 in accordance with embodiments of the present disclosure. As discussed above, position tracking device 114 may include any suitable device that may be able to locate and track foot 116 with any suitable technology. It should be noted that position tracking device 114 may be calibrated to operate and/or function in tracking area 700. Tracking area 700 may be a designated area in which position tracking device 114 may operate. For example, if foot 116 is outside tracking area 700, then position tracking device 114 may not record and/or track foot 116. It should be noted, that position tracking device 114 may record and/or track foot 116 outside of tracking area 700 but the movements may be disregarded by medical device 100. This may allow an operator to move foot 116 outside of tracking area 700 and not worry about medical device 100 operating an instrument 112 (e.g., referring to FIG. 1). Tracking area 700 may be further divided into zones. As illustrated in FIG. 7, tracking area 700 may comprise a first zone 702, a second zone 704, and/or a third zone 706. It should be noted that tracking area 700 may be calibrated for any number of suitable zones for a surgical operation. A zone may operate an individual surgical device. Thus, an operator may move foot 116 from one zone to another to operate different surgical devices. Once foot 116 is in an identified zone, the position and movement of foot 116 may be recorded and tracked by position tracking device 114. This information may be transmitted to medical console 102 wirelessly or through a wired connection for further processing by control system 110 (e.g., referring to FIG. 1).

It should be noted, that in embodiments tracking devices may be able to track light on the visible spectrum. This operation may described as motion tracking, time of flight, or video tracking. Without limitations, motion tracking or video tracking may utilize a single camera and/or multiple cameras working together. By detecting the movement of light across the lens of one and/or a plurality of camera. Tracing the movement of an operator's foot 116 may be translated into user input which may be feed into medical console 102. Examples of methods utilizing visible light may be time-of-flight tracking and/or 3D tracking.

Without limitation, 3-D tracking may use multiple cameras to record movement with visible light. Multiple cameras may be set-up at any angle relative to each other to determine the movement of an operator's foot 116 in space. Multiple cameras may also be used in time-of-flight tracking to determine the movement of operator's foot 116. Without limitation, cameras in a time-of-flight system may record the difference in speed of reflected light over time. In embodiments, a light source may be disposed in the camera or may be disposed within a suitable area outside of the camera. In examples, the camera and the light source may be synchronized. Synchronization may allow the camera to accurately record the time it may take light emanating from the light source to be recorded by the camera.

In embodiments, light may be pulsated out of the light source. As each pulse of light is reflect off an object, the camera may record the time it may take for the light to travel from the light source, reflect off the object, and be recorded by the camera. Changes in time for each light pulse may be calculated to determine movement of an object. It should be noted that the light source may be a laser.

In embodiments, position tracking device 114, may be a sensitive surface pad and may control a user input into console 102 (e.g., referring to FIG. 1). The sensitive surface pad may be disposed below an operator during a surgical procedure. Without limitations, the sensitive surface pad may be able to detect changes in pressure, resistance, capacity, and/or induction. For example, an operator may touch the sensitive surface pad with foot 116 (i.e., referring to FIG. 1). The sensitive surface pad may detect the pressure exerted on the sensitive surface pad from foot 116 through pressure sensors. In other embodiments, electrical means may be utilized to detect pressure change, and/or movement across the sensitive surface pad. For example, changes in electrical resistance across the sensitive surface pad may be measured, changes in capacitance, and/or changes in inductance may be measured to track movement of foot 116 across the sensitive surface pad. It should be noted that the sensitive surface pad may function and/or operate without pressure being applied as it may only sense change in electrical potential moving across the surface. This may allow the sensitive surface pad to determine position and movement of the position across the sensitive surface pad.

Medical console 102 may include a processor 708. Processor 708 may include any suitable device for processing instructions, including, but not limited to, a microprocessor, microcontroller, embedded microcontroller, programmable digital signal processor, or other programmable device. The processor 708 may also, or instead, be embodied in an application specific integrated circuit, a programmable gate array, programmable array logic, or any other device of combinations of devices operable to process electric signals. Processor 708 may also, or instead, be embodied in an application specific integrated circuit, a programmable gate array, programmable array logic, or any other device or combinations of devices operable to process electric signals. Processor 708 may be communicatively coupled to medical console 102. The connection between processor 708 and medical console 102 may be a wired connection or a wireless connection, as desired for a particular application. Processor 708 may be configured to receive user input 714, for example, to start and/or to stop the operation and/or function of instrument 112.

Medical console 102 may also include a memory 710, which may be internal or external, for example. Memory 710 may include any suitable form of data storage, including, but not limited to, electronic, magnetic, or optical memory, whether volatile or non-volatile. Memory 710 may include code 712 including instructions that may be executable by processor 708. Code 712 may be created, for example, using any suitable programming language, including but not limited to, C++ or any other programming language (including assembly languages, hardware description languages, and database programming languages) that may be stored, compiled, or interpreted to be executable by processor 708.

In operation, medical console 102 may receive the information about movement and/or position of foot 116 from position tracking device 114. For example, movement of foot 116 may be recorded and/or tracked by position tracking device 114, this information may be sent to medical console 102. The position of the foot from position tracking device 114 may be visualized on display screen 104, which may be a heads-up display or monitor. Display screen 104 may also provide feedback to the operator required to keep track of the current status and control options. Medical console 102 may receive this information from position tracking device 114, which may then be processed by processor 708. While not shown, processor 708 (or a different processor) may alternatively be integrated into position tracking device 114 so that processed data may be provided to medical console 102. Processor 708 may also receive information from user input 124. The information from the user input 124 may be in addition, or in place of, the information from position tracking device 114. Processor 708 may then process the information, from position tracking device 114, user input 716, or both position tracking device 114 and user input 716, to produce an output 714.

Output 714 may be defined as a set of commands that may be sent to an instrument 112. Commands in output 714 may direct instrument 112 to perform certain functions related to the surgical operation taking place. Instruction may be a simple as turning a light on and/or off or as complex as using instrument 112 on a patient. Commands in output 714 may originate from the movement of foot 116 or user input 716. For example, user input 716 may be functions and/or commands selected by an operator through display screen 104.

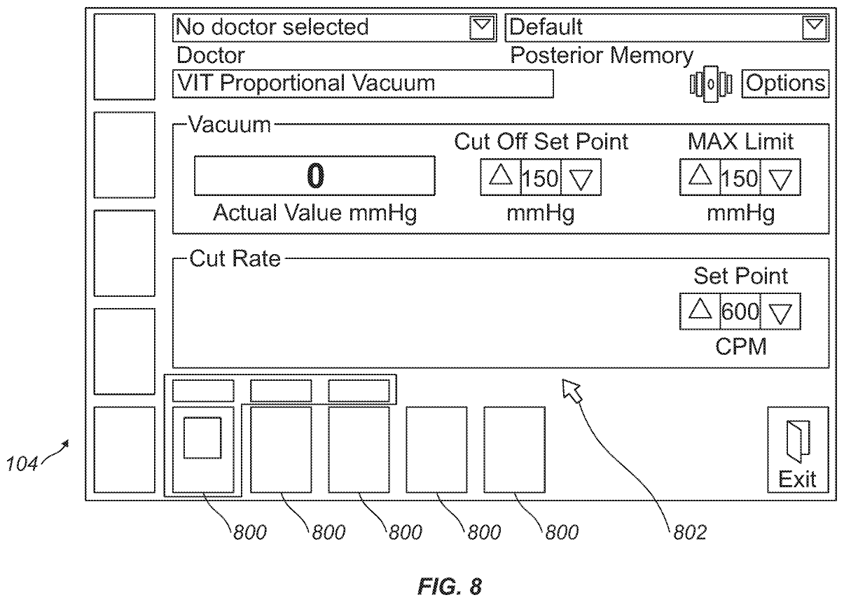

As illustrated in FIG. 8, display screen 104 may display patient information and/or status of instrument 112 (e.g., referring to FIG. 7). Display screen 104 may comprise a Graphical User Interface (GUI), which may be displayed on display screen 104, such that an operator may interact with medical console 102 (e.g., referring to FIGS. 1 and 7). In one embodiment, the GUI for medical device 100 may allow an operator to modally interact with medical console 102. In other words, the GUI may present an operator of medical console 102 a set of icons 800 or buttons corresponding to the entire range of functionality of medical console 102 or instrument 112 connected to medical console 102. Display screen 104 may allow an operator to select from these function icons 800 in order to utilize a particular functionality of medical console 102 or instrument 112. For example, an operator may use foot 116 (e.g., referring to FIG. 7) to select a surgical device through cursor 802. Cursor 802 may move as directed by an operators' foot 116 as foot 116 is tracked in tracking area 700 (e.g., referring to FIG. 7). In embodiments, a first zone 702, second zone 704, or third zone 706 may be designated as an area to access cursor 802. Foot 116 disposed in the particular zone tied to cursor 802 may allow the operator to move cursor 802, which may allow the operator to select different function on display screen 104.

Without limitations, icons 800 may be tied to an instrument 112. To access icons 800 an operator may dispose foot 116 into a first zone 702, second zone 704, or third zone 706 that may be tied to an icon 800. Icon 800 may further be tied to an instrument 112. This may allow operator to dispose foot 116 into the designated zone and issue commands through output 714 (e.g., referring to FIG. 7) to control instrument 112. Selecting icon 800 may alter display screen 104 by displaying a sub-command that may be specific to the selected instrument 112.

The operator may then configure any parameters or sub-modes for the desired functionality and utilize this functionality on instrument 112. Thus, during a surgical procedure, for each instrument 112 utilized in a surgical procedure an operator may interact with medical console 102 through position tracking device 114 or user input 716 to select the functionality desired for a surgical procedure and configure any parameters or sub-modes for instrument 112. For example, medical console 102 and/or instrument 112 may include functionality for vitreous cutting (Vit), vacuum (Extraction), Scissors, Viscous Fluid Control (VFC) and ultrasonic lens removal, an ophthalmic microscope, an ultrasonic handpiece, a surgery guidance system, and/or an intraoperative diagnostic unit. To implement a surgical procedure through medical console 102 and/or instrument 112, icon 800 may represent functionality desired of medical console 102 and/or instrument 112, and any parameters or sub-modes for that functionality configuration.

More specifically, embodiments of modes of interaction with a medical console 102 may be provided such that these modes of interaction limit or curtail the range of functionality which may be adjusted. In particular, certain embodiments may present one or more interfaces for operator interaction which may allow an operator to select from a set of pre-programmed options, where the interface or the set of preprogrammed options, icons 800, may correspond to the mode in which an operator may be interacting with medical console 102. Each of these preprogrammed options may correspond to settings for one or more parameters. By allowing an operator to select from a variety of preprogrammed options, the potential for mistakes and injury are reduced as the settings for each of the preprogrammed options may ensure that the settings for each of the parameters are proper relative to the settings for the other parameters and may similarly ensure that the values for certain parameters may not be set outside of a certain range. Additionally, as the set of parameters are adjusted in tandem according to preprogrammed settings, the interface for a particular mode of operation may be dramatically simplified relative to an interface which forces a doctor to adjust individually each parameter.

In embodiments, display screen 104 may be a touch screen, which may allow an operator to select icons 800 as user input 716 (e.g., Referring to FIG. 7). Specifically, the operator may select a mode of operation and/or instrument 112 by touching display screen 104. Based upon the mode of operation and/or instrument 112 selected, the GUI may present interface which presents an operator with the values of the current settings of a set of parameters. The interface may allow an operator to easily cycle through the set of preprogrammed options, for example by using a touch screen, position tracking device 114, and/or the like, and reflects changes to the settings of the parameters displayed which correspond to the currently selected preprogrammed option.

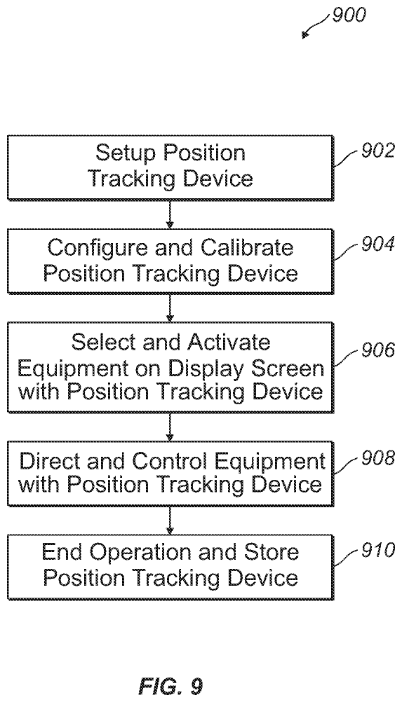

FIG. 9 illustrates a medical device control method 900 in accordance with embodiments of the present disclosure. As disclosed above, position tracking device 114 (e.g., referring to FIG. 1), may be used to control instrument 112 (e.g., referring to FIG. 7). To function and/or operate instrument 112 with position tracking device 114 in medical device control method 900, a first step 902 may be to setup position tracking device 114 for use with medical console 102. In embodiments, this may comprise determining a reasonable area for position tracking device 114 to be disposed. It should be noted that position tracking device 114 may comprise multiple components as described above. For example, position tracking device 114 may comprise a plurality of cameras. Each component may be disposed in an area where clutter and obstacles may not block the line of sight between the components and foot 116.

After setup, a second step 904 may be to configure and calibrate position tracking device 114. Second step 904 may calibrate and/or configure position tracking device 114 to operate and/or function an area selected in first step 902. For example, calibration and/or configuration may identify the boundaries to tracking area 700 and the number of zones disposed in tracking area 700. Each zone may control the functionality and/or operation of medical device 100. During second step 904, the number of zones may be selected and identifies in tracking area 700. After second step 904, position tracking device 114 may be utilized in a surgical operation. Depending on the particular position tracking device 114, calibration may or may not be required.

In the third step 906, an operator may select an instrument 112 using position tracking device 114. For example, an operator may identify an instrument 112 to operate and/or function. The operator may move foot 116 into tracking area 700 and into a specified zone that may control the identified instrument 112. As foot 116 is disposed in the zone, display screen 104 may display an icon 800 that may be associated with the identified instrument 112. Operator may move and/or position foot 116 in a designated area of the zone to activate the instrument 112. Alternatively, operator may use to foot 116 to select a particular instrument 112 on display screen 104. Further movements of foot 116 may activate certain functions and/or operations of instrument 112.

In fourth step 908, instrument 112 may operate and/or function as controlled by foot 116 through position tracking device 114. Functions and/or operations of instrument 112 may be repeated any number of times through movement of foot 116. Additionally, other medical devices may be selected by moving foot 116 from one zone to another zone or by selecting the other instrument 112 on display screen 104. Display screen 104 may also provide feedback to the operator to keep track of the current status and control options. Visual feedback from display screen 104 may indicated to the operator which zone foot 116 may be disposed.

In fifth step 910, the operator may remove foot 116 from tracking area 700, which may prevent operation and/or function of instrument 112 through position tracking device 114. Position tracking device 114 may be stored with medical console 102 for future use in future operations.

It is believed that the operation and construction of the present disclosure will be apparent from the foregoing description. While the apparatus and methods shown or described above have been characterized as being preferred, various changes and modifications may be made therein without departing from the spirit and scope of the disclosure as defined in the following claims.

What is claimed is:

1. A system, comprising:
at least one medical device;
a display screen coupled to the medical device; and
a virtual foot pedal, wherein the virtual foot pedal is connected to the medical device and operable to capture motion in a tracking area.

2. The system of claim 1, wherein the tracking area comprises at least one zone.

3. The system of claim 2, wherein the at least one zone controls the at least one medical device.

4. The system of claim 1, wherein the display screen is a heads up display and comprises at least one icon that controls the at least one medical device.

5. The system of claim 4, wherein the at least one icon is operable to access sub-modes, wherein the sub-modes are operable to control functionality of the at least one medical device.

6. The system of claim 1, wherein the medical device further comprises a console, wherein the virtual foot pedal is wirelessly connected to the console.

7. The system of claim 1, wherein the medical device further comprises a console, wherein the virtual foot pedal is wired to the console.

8. The system of claim 1, wherein the virtual foot pedal comprises a camera, wherein the camera comprises a light source, wherein the light source is pulsated.

9. The system of claim 1, wherein the virtual foot pedal comprises a camera and markers, wherein the markers disposed on a foot, wherein the camera is operable to track movement of the markers in the tracking area.

10. The system of claim 9, wherein the camera is operable to sense visible light.

11. The system of claim 1, wherein the virtual foot pedal comprises an infrared light source, wherein the infrared light source is operable to emit an infrared light into the tracking area.

12. The system of claim 1, wherein the virtual foot pedal comprises:
a body, wherein the body is capable of stabilizing the virtual foot pedal and wherein the body further comprises a transmitter and a receiver.

13. The system of claim 12, wherein the transmitter is operable to emit a sound wave into the tracking area.

14. The system of claim 13, wherein the receiver is operable to sense a reflected wave from the tracking area.

15. The system of claim 12, wherein the transmitter is operable to emit an electromagnetic field into the tracking area.

16. The system of claim 15, wherein the virtual foot pedal further comprises magnets, wherein the magnets are positionable in the tracking area and the receiver is operable to sense alterations to the electromagnetic field due to the magnets.

17. A method for controlling a medical console comprising:
displaying one or more icons disposed on a display screen disposed on the medical console;
initiating a virtual foot pedal to emit a tracking area;
monitoring movement of a foot in the tracking area; and
receiving a selection of at least one icon based on movement of the foot.

18. The method of claim 17, further comprising operating a medical device with the foot.

19. The method of claim 17, wherein the virtual foot pedal is attached to the medical console wirelessly.

20. The method of claim 17, wherein the at least one icon is operable to access sub-modes, wherein the sub-modes are operable to control functionality of the medical console.
